(19) United States
(12) Patent Application Publication
(10) Pub. No.: US 2018/0014904 A1
(43) Pub. Date: Jan. 18, 2018

(54) OPTICAL AND DIGITAL VISUALIZATION IN A SURGICAL MICROSCOPE

(71) Applicant: Novartis AG, Basel (CH)

(72) Inventors: Lingfeng Yu, Rancho Santa Margarita, CA (US); Hugang Ren, Cypress, CA (US)

(21) Appl. No.: 15/617,870

(22) Filed: Jun. 8, 2017

Related U.S. Application Data
(60) Provisional application No. 62/361,227, filed on Jul. 12, 2016.

(51) Int. Cl. H04N 5/235; G02B 21/00; G02B 21/04; H04N 5/232; G02B 21/36

(52) U.S. Cl. CPC A61B 90/37; H04N 5/2353; H04N 5/23293; G02B 21/368; G02B 21/367; A61B 90/20; G02B 21/04; G02B 21/0012; G02B 21/361; A61B 90/361; A61B 2090/373

(57) ABSTRACT

A surgical microscope may support intraoperative viewing of optical and digital images of a surgical site during surgery. The optical images may be viewed using an optical beam path from an objective lens of the surgical microscope. The digital images may be generated by redirecting or splitting the optical beam path to an imaging system that outputs digital data representing the digital image to a display for output to the user, such as through an ocular of the surgical microscope.

### BACKGROUND

### Field of the Disclosure

The present disclosure relates to ophthalmic surgery, and more specifically, to optical and digital visualization in a surgical microscope.

### Description of the Related Art

In ophthalmology, eye surgery, or ophthalmic surgery, saves and improves the vision of tens of thousands of patients every year. However, given the sensitivity of vision to even small changes in the eye and the minute and delicate nature of many eye structures, ophthalmic surgery is difficult to perform and the reduction of even minor or uncommon surgical errors or modest improvements in accuracy of surgical techniques can make an enormous difference in the patient's vision after the surgery.

Ophthalmic surgery is performed on the eye and accessory visual structures. More specifically, vitreoretinal surgery encompasses various delicate procedures involving internal portions of the eye, such as the vitreous humor and the retina. Different vitreoretinal surgical procedures are used, sometimes with lasers, to improve visual sensory performance in the treatment of many eye diseases, including epimacular membranes, diabetic retinopathy, vitreous hemorrhage, macular hole, detached retina, and complications of cataract surgery, among others. During vitreoretinal surgery, for example, an ophthalmologist typically uses a surgical microscope to view the fundus through the cornea, while surgical instruments that penetrate the sclera may be introduced to perform any of a variety of different procedures. The surgical microscope provides imaging and optionally illumination of the fundus during vitreoretinal surgery.

In other types of surgery, the surgical microscope may be used with various surgical sites during surgical procedures.

Typical surgical microscopes are equipped with analog optical systems for viewing intraoperative images formed from light transmitted through an objective lens of the microscope.

### SUMMARY

The disclosed embodiments of the present disclosure provide for optical and digital visualization using a surgical microscope. The optical and digital visualization using a surgical microscope disclosed herein may enable a user of the surgical microscope to select either an analog image or a digital image for viewing during surgery. In this manner, enhancements enabled by digital imaging, such as control of image brightness, contrast, as well as digital annotations added to the field of view, may be provided for intraoperative viewing.

In one aspect, a disclosed method for displaying images during surgery includes displaying an analog image of a surgical site to a user using a surgical microscope. In the method, the analog image may include light from an objective lens of the surgical microscope. The method may include receiving a first indication to display a digital image of the surgical site using the surgical microscope. Responsive to the first indication, the method may include redirecting the light from the objective lens to an imaging system enabled to acquire the digital image, and displaying the digital image of the surgical site to the user using a display device.

In any of the disclosed embodiments, the method may further include performing digital processing on the digital image. In the method, the digital processing may include at least one operation selected from changing the contrast of the digital image, changing the brightness of the digital image, annotating the digital image with text annotations, annotating the digital image with a measurement display, and annotating the digital image with a digital marker.

In any of the disclosed embodiments, the method may further include receiving a second indication to display the analog image of the surgical site using the surgical microscope. Responsive to the second indication, the method may include redirecting the light from the objective lens to an ocular of the surgical microscope, and displaying the analog image in the ocular.

In any of the disclosed embodiments of the method, redirecting light from the objective lens may further include controlling a first mirror shutter in an optical path of the light to redirect the light to the imaging system, and controlling a second mirror shutter in the optical path to redirect the digital image to the ocular.

In any of the disclosed embodiments of the method redirecting light to the ocular may further include controlling a first mirror shutter in an optical path of the light to redirect the light to the ocular, and controlling a second mirror shutter in the optical path to redirect the light to the ocular.

In any of the disclosed embodiments of the method, redirecting light from the objective lens may further include using a beam splitter in an optical path of the light to split the light between the imaging system and the optical path, using a beam combiner in the optical path to superimpose the digital image to the optical path, controlling a shutter between the beam splitter and the beam combiner to control propagation of the light from the objective lens, and controlling power to the display device for displaying the digital image.

In another aspect, a surgical microscope for displaying images during surgery is disclosed. The surgical microscope may include a controller enabled to control redirection of light from an objective lens of the surgical microscope for display to a user. In the surgical microscope, the controller may be further enabled to display an analog image of a surgical site to the user. In the surgical microscope, the analog image may include light from the objective lens. In the surgical microscope, the controller may be further enabled to receive a first indication to display a digital image of the surgical site. Responsive to the first indication, the controller may be further enabled to redirect the light from the objective lens to an imaging system enabled to acquire the digital image, and display the digital image of the surgical site to the user using a display device.

In any of the embodiments of the surgical microscope, the controller may be further enabled to perform digital processing on the digital image, the digital processing including at least one operation selected from changing the contrast of the digital image, changing the brightness of the digital image, annotating the digital image with text annotations, annotating the digital image with a measurement display, and annotating the digital image with a digital marker.

In any of the embodiments of the surgical microscope, the controller may be further enabled to receive a second indication to display the analog image of the surgical site using the surgical microscope. Responsive to the second indication, the controller may be further enabled to redirect the light to an ocular of the surgical microscope, and display the analog image in the ocular.

In any of the embodiments of the surgical microscope, redirecting light from the objective lens may further include controlling a first mirror shutter in an optical path of the light to redirect the light to the imaging system, and controlling a second mirror shutter in the optical path to redirect the digital image to the ocular.

In any of the embodiments of the surgical microscope, redirecting light to the ocular may further include controlling a first mirror shutter in an optical path of the light to redirect the light to the ocular, and controlling a second mirror shutter in the optical path to redirect the light to the ocular.

In any of the embodiments of the surgical microscope, redirecting light from the objective lens may further include using a beam splitter in an optical path of the light to split the light between the imaging system and the optical path, using a beam combiner in the optical path to superimpose the digital image to the optical path, controlling a shutter between the beam splitter and the beam combiner to control propagation of the light from the objective lens, and controlling power to the display device for displaying the digital image.

In yet a further aspect, a controller for a surgical microscope for displaying images during surgery is disclosed. The controller may be enabled to display an analog image of a surgical site to the user, where the analog image comprises light from the objective lens. The controller may further be enabled to receive a first indication to display a digital image of the surgical site. Responsive to the first indication, the controller may further be enabled to redirect the light from the objective lens to an imaging system enabled to acquire the digital image, and display the digital image of the surgical site to the user using a display device.

In any of the disclosed embodiments, the controller may further be enabled to perform digital processing on the digital image, the digital processing including at least one operation selected from changing the contrast of the digital image, changing the brightness of the digital image, annotating the digital image with text annotations, annotating the digital image with a measurement display, and annotating the digital image with a digital marker.

In any of the disclosed embodiments, the controller may further be enabled to receive a second indication to display the analog image of the surgical site using the surgical microscope. Responsive to the second indication, the controller may further be enabled to redirect the light to an ocular of the surgical microscope, and display the analog image in the ocular.

In any of the disclosed embodiments of the controller, redirecting light from the objective lens may further include controlling a first mirror shutter in an optical path of the light to redirect the light to the imaging system, and controlling a second mirror shutter in the optical path to redirect the digital image to the ocular.

In any of the disclosed embodiments of the controller, redirecting light to the ocular may further include controlling a first mirror shutter in an optical path of the light to redirect the light to the ocular, and controlling a second mirror shutter in the optical path to redirect the light to the ocular.

In any of the disclosed embodiments of the controller, redirecting light from the objective lens may further include using a beam splitter in an optical path of the light to split the light between the imaging system and the optical path, using a beam combiner in the optical path to superimpose the digital image to the optical path, controlling a shutter between the beam splitter and the beam combiner to control propagation of the light from the objective lens, and controlling power to the display device for displaying the digital image.

### BRIEF DESCRIPTION OF THE DRAWINGS

For a more complete understanding of the present invention and its features and advantages, reference is now made to the following description, taken in conjunction with the accompanying drawings, in which:

FIG. 1A is a block diagram of selected elements of an embodiment of a surgical microscopy with analog and digital display;

FIG. 1B is a block diagram of selected elements of an embodiment of a surgical microscopy with analog and digital display;

FIG. 2 is an embodiment of a surgical microscopy digital display;

FIG. 3 is a block diagram of selected elements of an embodiment of a controller; and

FIG. 4 is a flow chart of selected elements of a method for optical and digital visualization in a surgical microscope.

### DETAILED DESCRIPTION

In the following description, details are set forth by way of example to facilitate discussion of the disclosed subject matter. It should be apparent to a person of ordinary skill in the field, however, that the disclosed embodiments are exemplary and not exhaustive of all possible embodiments.

As used herein, a hyphenated form of a reference numeral refers to a specific instance of an element and the un-hyphenated form of the reference numeral refers to the collective element. Thus, for example, device ‘12-1’ refers to an instance of a device class, which may be referred to collectively as devices ‘12’ and any one of which may be referred to generically as a device ‘12’.

As noted above in one example of a surgery and a surgical site, surgical microscopes are used during ophthalmic surgery to view the eye of the patient. For example, during vitreoretinal surgery a surgeon may view the fundus of an eye of a patient using a surgical microscope, for example, in conjunction with a contact lens placed on the cornea. In other instances, the surgical microscope may be used to view the cornea, iris, and lens, such as during cataract surgery. Typical surgical microscopes provide an analog image formed from light transmitted through an objective lens of the surgical microscope. Although analog images are well known and offer some flexibility with regard to optical performance, such as a wide dynamic range of illumination, high resolution, and good depth perception, analog images cannot provide various enhancements that digital images provide for intraoperative viewing. Digital images are formed as light is transmitted from the objective lens to an imaging system having a light sensitive sensor (a camera), such as a charge coupled device (CCD) array of optical sensors. The light sensitive sensor transforms the digital image into digital data, which can be processed using various methods, and which can subsequently be sent to a display for viewing of the digital image by the user of the surgical microscope.

The present disclosure relates to optical and digital visualization in a surgical microscope. The methods and systems disclosed herein for optical and digital visualization in a surgical microscope may enable a user to select between an analog image and a digital image for intraoperative viewing during surgery. The methods and systems disclosed herein for optical and digital visualization in a surgical microscope may enable digital image processing of the digital image prior to view to provide for various image enhancements that are not available with analog images alone. The methods and systems disclosed herein for optical and digital visualization in a surgical microscope may enable digital image processing for changing the contrast of the digital image, changing the brightness of the digital image, annotating the digital image with text annotations, annotating the digital image with a measurement display, and annotating the digital image with a digital marker. The methods and systems disclosed herein for optical and digital visualization in a surgical microscope may further enable detection, extraction, or enhancement of features in the digital image, such as tissue structures at a surgical site, that may be difficult or impossible to observe in the analog image alone. In this manner, the methods and systems disclosed herein for optical and digital visualization in a surgical microscope may provide significant improvement in the information and quality of images viewed intraoperatively during surgery.

As will be described in further detail, optical and digital visualization in a surgical microscope may be realized using a pair of mirror shutters in an ocular beam path from the objective lens. A first mirror shutter may enable light from the objective lens to be directed either towards the ocular (to view the analog image) or towards an imaging system (to generate the digital image). The imaging system may perform various image enhancements and annotations after the digital image is acquired as digital data. The imaging system may then output the digital image (in the form of digital data) to a display that is viewable from the ocular. A second mirror shutter may enable either light from the objective lens to be directed towards the ocular (to view the analog image) or light from a display to be directed towards the ocular (to output the digital image). Alternatively, a beam splitter and a beam combiner may be used in conjunction with a shutter in between to either select the optical image, the digital image, or a superimposition of the optical image and the digital image. In various embodiments, the surgical microscope may be designed with binocular optics, such that a left ocular beam and a right ocular beam are each respectively enabled to display either the analog image or the digital image in a left ocular and a right ocular simultaneously, as will be described in further detail below.

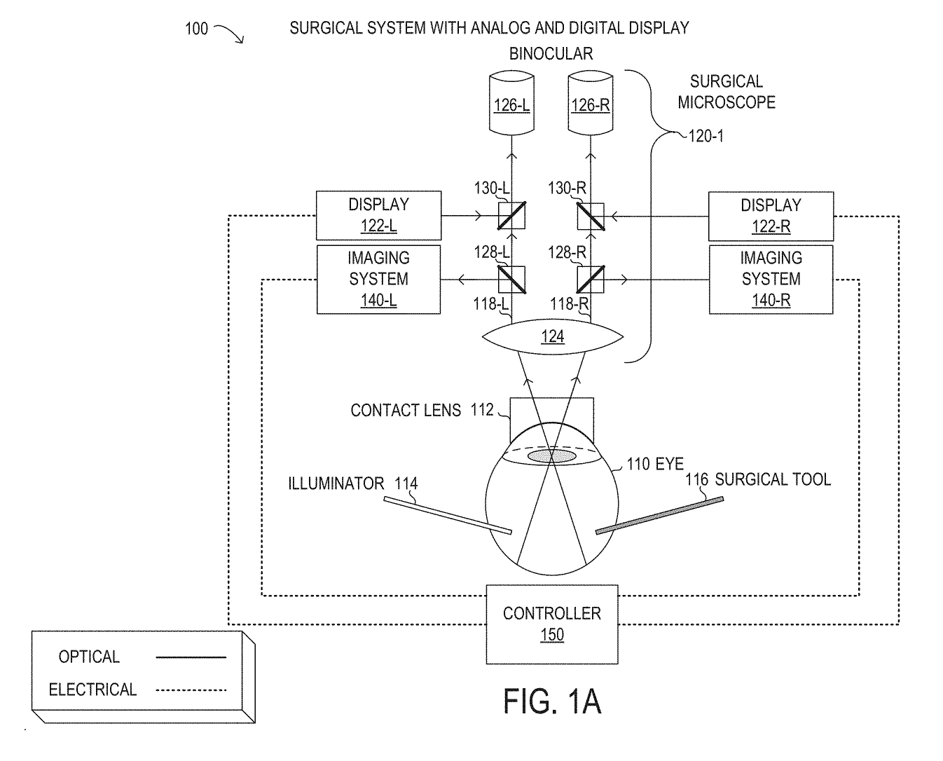

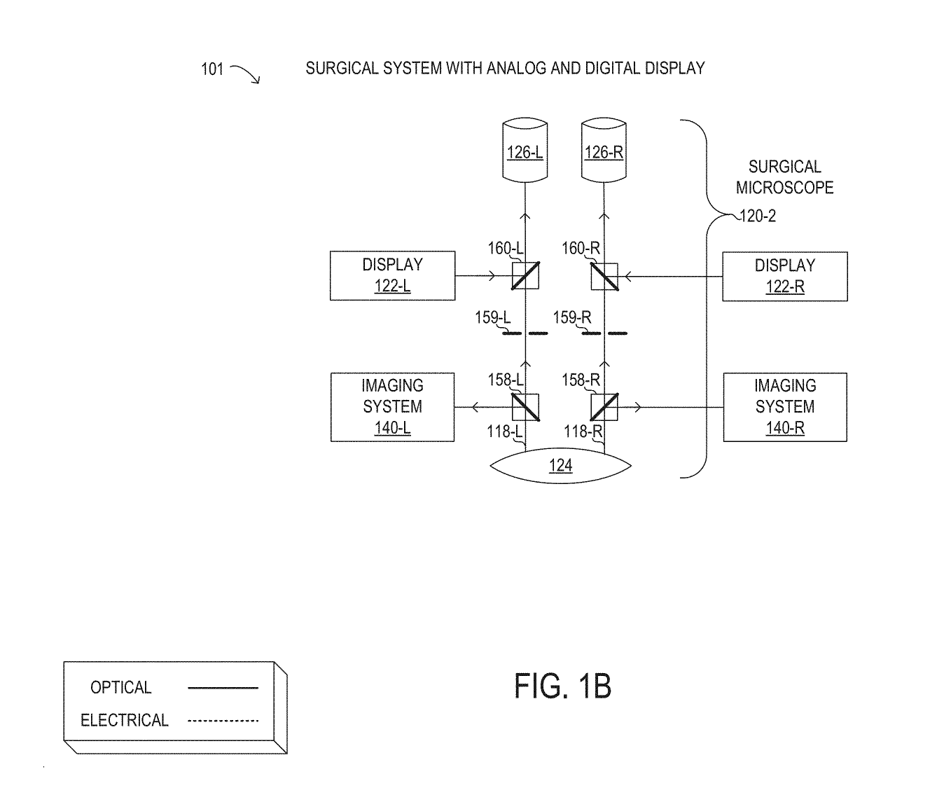

Referring now to the drawings, FIG. 1A is a block diagram showing a surgical system with analog and digital display 100. Surgical system 100 is not drawn to scale but is a schematic representation. As will be described in further detail, surgical system 100 may be used during ophthalmic surgery to view and analyze a human eye 110 with either analog images or digital images. Although surgical microscope 120 is shown for use in ophthalmic surgery with the eye as the surgical site, it will be understood that the methods and systems disclosed herein may be applied to any kind of surgery and surgical site where surgical microscopy is used. As shown, surgical system 100 includes surgical microscope 120-1, controller 150, imaging system 140, display 122, as well as mirror shutters 128, 130, binoculars 126, and objective lens 124. It is noted that FIG. 1A depicts an exemplary arrangement of surgical system 100 for vitreoretinal surgery with contact lens 112 enabling view of the fundus, where surgical tool 116 and illuminator 114 may be used. In various embodiments, it will be understood, for example, that contact lens 112 may be omitted and surgical system 100 may be used to directly view the cornea and associated structures of eye 110.

As shown, surgical microscope 120-1 is depicted in schematic form to illustrate optical functionality. It will be understood that surgical microscope 120-1 may include various other optical, electronic, mechanical components, in different embodiments. For example, various lenses and optical elements along ocular path 118 have been omitted from FIG. 1A for descriptive clarity. Accordingly, objective lens 124 may represent a selectable objective to provide a desired magnification or field of view of eye 110. Objective lens 124 may receive light from eye 110 that may be generated by surgical microscope 120-1, or another source, such as illuminator 114, in various embodiments. As shown in FIG. 1A, a left ocular beam 118-L and a right ocular beam 118-R may be formed from light emerging from eye 110, such as light reflected back from a light source (not shown) that transmits incident light through objective lens 124. It is noted that the light source may be the same for generating analog images or digital images for viewing with surgical microscope 120-1.

In FIG. 1A, surgical microscope 120-1 is shown with a binocular arrangement with two distinct but substantially equal light paths, namely left ocular beam 118-L and right ocular beam 118-R, that enable viewing with binoculars 126 that comprise a left ocular 126-L and a right ocular 126-R. From objective lens 124, left ocular beam 118-L may arrive at mirror shutter 128-L, which is controllable to either reflect left ocular beam 118-L to imaging system 140-L or to pass left ocular beam 118-L towards left ocular 126-L. When mirror shutter 128-L passes left ocular beam 118-L, left ocular beam 118-L arrives at mirror shutter 130-L, which is controllable to either pass left ocular beam 118-L towards left ocular 126-L or to reflect output light from display 122-L to left ocular 126-L. Similarly, from objective lens 124, right ocular beam 118-R may arrive at mirror shutter 128-R, which is controllable to either reflect right ocular beam 118-R to imaging system 140-R or to pass right ocular beam 118-R towards right ocular 126-R. When mirror shutter 128-R passes right ocular beam 118-R, right ocular beam 118-R arrives at mirror shutter 130-R, which is controllable to either pass right ocular beam 118-R towards right ocular 126-R or to reflect output light from display 122-R to right ocular 126-R. Accordingly in surgical microscope 120-1, when mirror shutters 128, 130 pass respective ocular beam 118, the analog image of eye 110 is viewed at binoculars 126, and when mirror shutters 128, 130 reflect respective ocular beam 118, the digital image of eye 110 is viewed at binoculars 126. It is noted that imaging system 140 may be a singular system supporting left and right beams and is shown as 140-L and 140-R for descriptive clarity in FIG. 1A.

Display 122 may represent a digital display device, such as a liquid crystal device (LCD) array. Display 122-L may generate a digital image for left ocular 126-L, while display 122-R may generate a digital image for right ocular 126-R. In some embodiments, display 122 includes miniature display devices that output images to binoculars 126 for viewing by the user and are integrated within the ocular optics of surgical microscope 120-1. It is noted that display 122 may be a singular device with separate left and right display regions and is shown as 122-L and 122-R for descriptive clarity in FIG. 1A.

In FIG. 1A, controller 150 may have an electrical interface with display 122, for example, sending digital data indicative of the digital image (not shown). In this manner, controller 150 may receive digital data indicative of the digital image from imaging system 140, may modify the digital data as described herein, and may output the digital image to display 122 that is viewed at binoculars 126. Because the electrical interface between imaging system 140, display 122 and controller 150 may support digital data processing, controller 150 may perform image processing on the digital data in real-time with relatively high frame refresh rates, such that a user of surgical microscope 120-1 may experience substantially instantaneous display with little or no latency. Display 122 may comply with a display standard for the corresponding type of display, such as video graphics array (VGA), extended graphics array (XGA), digital visual interface (DVI), high-definition multimedia interface (HDMI), etc.

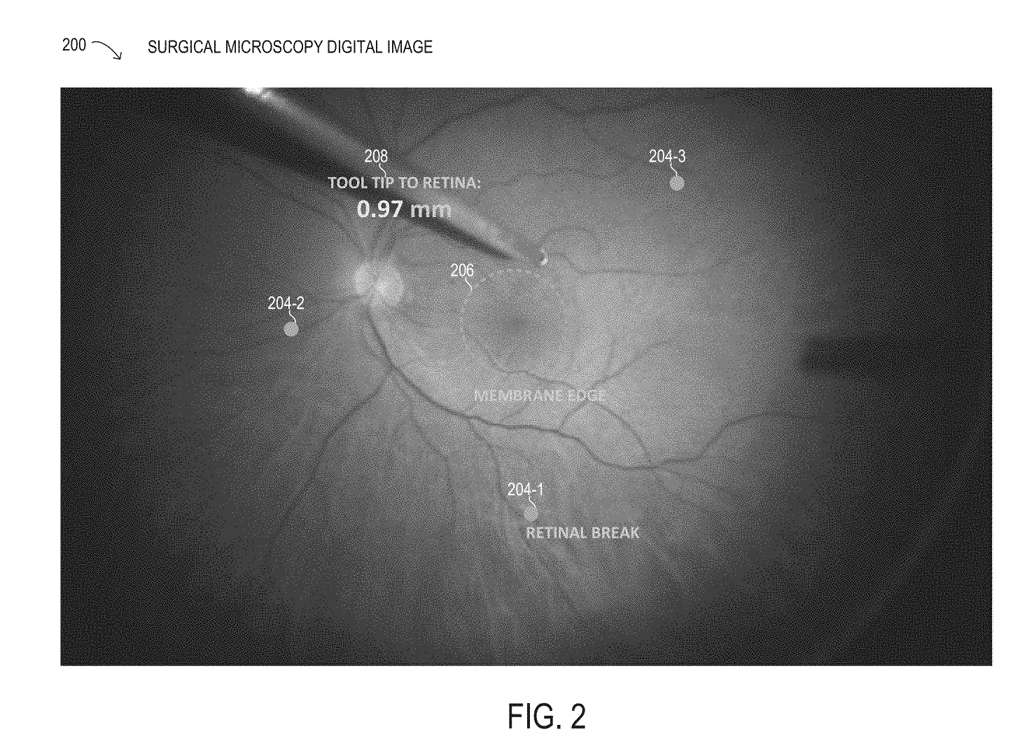

With the arrangement of surgical microscope 120-1 in FIG. 1A, imaging system 140 may represent any of a variety of different kinds of imaging systems, as desired. Imaging system 140 may acquire digital images as light is transmitted from objective lens 124 to imaging system 140. Imaging system 140 may have a light sensitive sensor (a camera), such as a charge coupled device (CCD) array of optical sensors. The light sensitive sensor transforms the digital image into digital data, which can be processed using various methods, and which can subsequently be sent to display 122 for viewing of the digital image by the user of surgical microscope 120-1. As noted, imaging system 140 may perform various kinds of digital processing on the digital image. In particular embodiments and as described in further detail with regard to FIG. 2, the digital processing performed by imaging system 140 may include any one or more of: changing the contrast of the digital image, changing the brightness of the digital image, annotating the digital image with text annotations, annotating the digital image with a measurement display, and annotating the digital image with a digital marker.

In operation of surgical system 100, initially, mirror shutters 128, 130 may be set to pass through light from objective lens 124 to binoculars 126 along ocular beam path 118 to enable viewing of the analog image. A user may accordingly view analog images of eye 110, such as intraoperatively during ophthalmic surgery, using surgical microscope 120-1. The user may then provide a first indication, such as a command (using a button, software command, voice command, gesture, etc.), to controller 150 to display the digital image. Controller 150 may then cause mirror shutters 128, 130 to actuate to place a mirror in the ocular beam paths 118. As a result, light from objective lens 124 is redirected to imaging system 140, where the light is acquired and digitally sampled to generate the digital image in the form of digital data. As directed or set-up by the user, imaging system 140 may further perform digital image processing on the image, as desired. After any digital image processing (or no digital image processing) is performed, the resulting digital image in the form of digital data may be sent to display 122, which outputs the digital image to binoculars 126 for viewing by the user. Additionally, at some later time, the user may provide a second indication to switch surgical microscope 120-1 back to viewing with the analog image, such that mirror shutters 128, 130 are once again set to pass through light along ocular beam path 118 to binoculars 126, as described above. In this manner, the user may be enabled to switch back and forth between viewing the analog image and the digital image.

Modifications, additions, or omissions may be made to surgical microscopy scanning instrument 100 without departing from the scope of the disclosure. The components and elements of surgical system 100, as described herein, may be integrated or separated according to particular applications. Surgical system 100 may be implemented using more, fewer, or different components in some embodiments.

FIG. 1B is a block diagram showing a surgical system with analog and digital display 101. Surgical system 101 is not drawn to scale but is a schematic representation. Surgical system 101 shows a second embodiment of surgical microscope 120-2 that may be used in surgical system 100 in place of surgical microscope 120-1 described above, while various other details of surgical system 100 have been omitted in surgical system 101 for descriptive clarity.

Specifically, in surgical microscope 120-2, for example, ocular beam 118-L from objective lens 124 may pass through a beam splitter 158-L, which may split ocular beam 118-L into two beams: a first beam to imaging system 140-L and the remaining light propagating along ocular beam 118-L. In one example, 30% of the intensity is directed to the first beam while 70% of the intensity remains in ocular beam 118-L. A beam combiner 160-L may receive the digital image from display 122-L and superimpose the digital image onto ocular beam 118-L. Additionally, a shutter 159-L may control propagation of ocular beam 118-L from beam splitter 158-L. A similar arrangement with beam splitter 158-R, shutter 159-R, and beam combiner 160-R may be implemented for the right ocular in surgical microscope 120-2. Thus, in surgical microscope 120-2, the optical image may be viewed by opening shutter 159 and turning off display 122. In surgical microscope 120-2, the digital image may be viewed by closing shutter 159 and turning on display 122. In surgical microscope 120-2, the optical image and the digital image may be superimposed by opening shutter 159 and turning on display 122.

In surgical microscope 120-2, shutter 159 may be a manual shutter, while power to turn on display 122 and imaging system 140 may also be manually controlled in some embodiments. In particular embodiments, however, shutter 159 may be a servo-mechanical shutter that is controlled by controller 150, while display 122 may also be powered on or off under control of controller 150. Additionally, power to imaging system 122 may also be under control of controller 150, for example, to control whether the digital image is acquired or not. For example, when an indication is received at controller 150 to redirect light to the imaging system, the imaging system may be enabled to acquire the digital image by being powered on, or a check may be performed that imaging system is powered on and enabled to acquire the digital image.

FIG. 2 shows an embodiment of a surgical microscopy digital image 200. Digital image 200 may represent a field of view seen by the user operating surgical system 100 (see FIG. 1A) when the digital image is selected, as described above with respect to FIGS. 1A or 1B. As shown, display image 200 may be enhanced in various aspects as compared to the analog image (not shown). Specifically, the contrast and brightness of the digital image may be changed or modified to a user-selected value or range of values. Other properties of digital image 200 may also be modified, such as a color palette specifying colors used to display various intensities and color values in the digital data comprising the digital image. In other words, a color mapping according to any desired color palette may be used, including greyscale, or other monochromatic color palette using any arbitrary color instead of grey.

In FIG. 2, various annotations to the digital image are shown, which may be generated by controller 150 by digital image processing, as mentioned previously. Specifically, markers 204-1 may be shown at locations of desired features, such as marker 204-1 that is annotated with the text “RETINAL BREAK” to show the location of a retinal break. Other markers 204-2 and 204-3 may be associated, either manually or using auto-detection of tissue structures, with various features, such as blood vessels, membranes, or other relevant features. Also, region 206 shows an example of a regional annotation that is annotated with the text “MEMBRANE EDGE” to show the edges of a membrane, such as the epiretinal membrane (ERM). Furthermore, certain annotations may represent actual measurements performed on the eye, such as measurement 208, which is an annotation comprising the text “TOOL TIP TO RETINA: 0.97 mm” where the distance of a tool tip to the retina is measured and displayed in real-time in digital image 200. It is noted that other types of annotations and markers may be used in various embodiments.

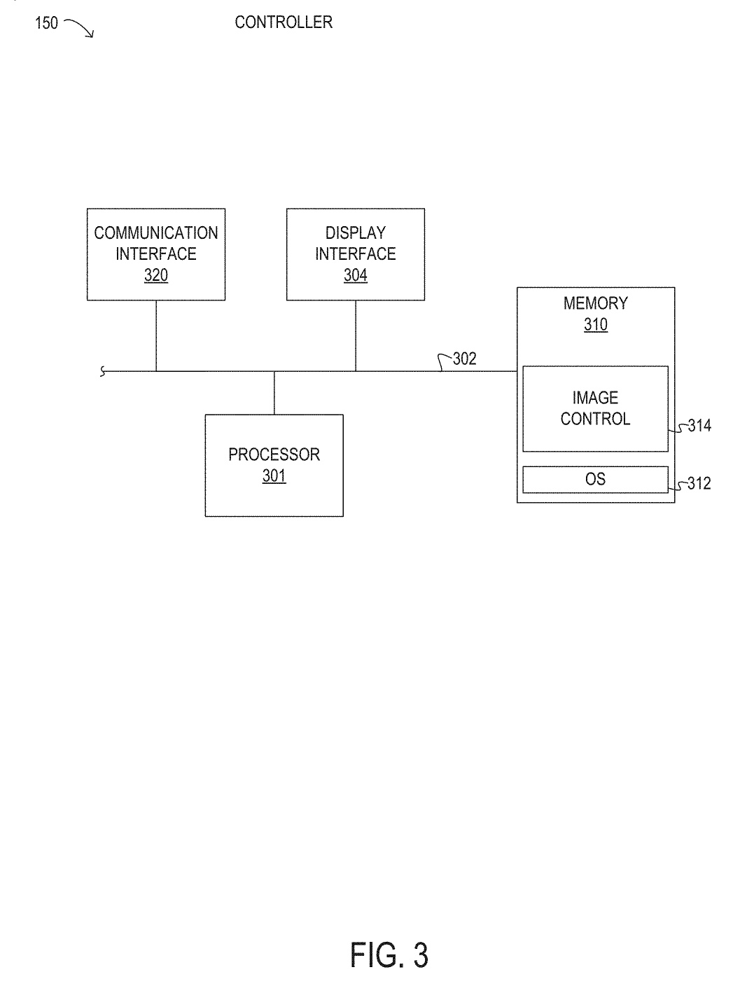

Referring now to FIG. 3, a block diagram illustrating selected elements of an embodiment of controller 150, described above with respect to FIG. 1A, is presented. In the embodiment depicted in FIG. 3, controller 150 includes processor 301 coupled via shared bus 302 to memory media collectively identified as memory 310.

Controller 150, as depicted in FIG. 3, further includes communication interface 320 that can interface controller 150 to various external entities, such as imaging system 140 and display 122, among others. In some embodiments, communication interface 320 is operable to enable controller 150 to connect to a network (not shown in FIG. 3). In embodiments suitable for optical and digital visualization in a surgical microscope, controller 150, as depicted in FIG. 3, may include display interface 304 that connects shared bus 302, or another bus, with an output port for one or more displays, such as display 122 or an external display (not shown).

In FIG. 3, memory 310 encompasses persistent and volatile media, fixed and removable media, and magnetic and semiconductor media. Memory 310 is operable to store instructions, data, or both. Memory 310 as shown includes sets or sequences of instructions, namely, an operating system 312, and an image control application 314. Operating system 312 may be a UNIX or UNIX-like operating system, a Windows® family operating system, or another suitable operating system. Image control application 314 may perform digital image processing, as describe herein.

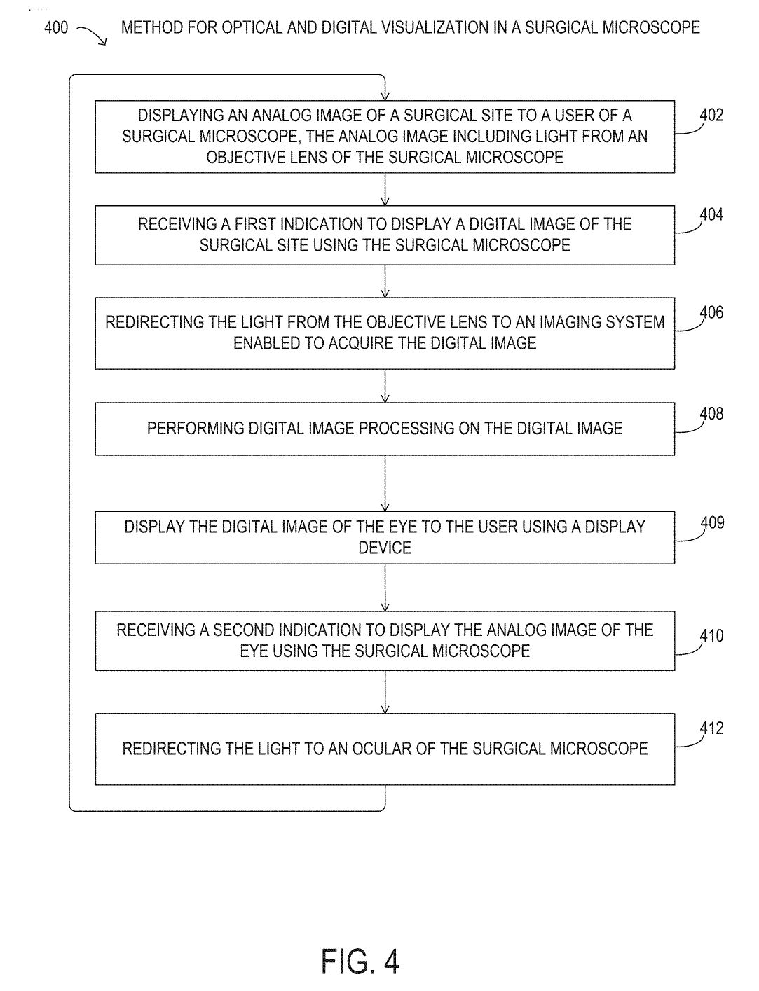

Referring now to FIG. 4, a flow chart of selected elements of an embodiment of a method 400 for optical and digital visualization in a surgical microscope, as described herein, is depicted in flowchart form. Method 400 describes steps and procedures that controller 150 (or image control application 314) may perform while a user operates surgical system 100. It is noted that certain operations described in method 400 may be optional or may be rearranged in different embodiments. The user referred to in method 400 may be a surgeon or other medical personnel. In some embodiments, at least certain portions of method 400 may be automated, for example using servo-mechanical control associated with certain aspects of surgical microscope 120-1, such as activating mirror shutters 128, 130, controlling power to display 122 or to imaging system 140, and activating shutter 159 in surgical microscope 120-2.

Method 400 may begin, at step 402, by displaying an analog image of surgical site to a user of a surgical microscope, the analog image including light from an objective lens of the surgical microscope. At step 404, a first indication to display a digital image of the surgical site using the surgical microscope is received. The first indication at step 404 may represent user input provided by the user. At step 406, the light from the objective lens is redirected to an imaging system enabled to acquire the digital image. In some embodiments, such as shown in FIG. 1B, the first indication may be used to control power to imaging system 140, thereby enabling imaging system 140 to acquire the digital image. At step 408, digital image processing is performed on the digital image. At step 409, the digital image of the eye is displayed to the user using a display device. The display device in step 409 may output the digital image to an ocular of the surgical microscope. At step 410, a second indication to display the analog image of the eye using the surgical microscope is received. The second indication at step 410 may represent user input provided by the user. At step 412, the light is redirected to an ocular of the surgical microscope. In some embodiments, such as shown in FIG. 1B, instead of redirecting the light, shutter 159 as well as power to display 122 may be controlled to display the optical image in the ocular. After 412, method 400 loops back to operation 402. It is noted that method 400 may wait at corresponding steps to receive the first indication or the second indication.

As disclosed herein, a surgical microscope may support intraoperative viewing of optical and digital images of a surgical site during surgery. The optical images may be viewed using an optical beam path from an objective lens of the surgical microscope. The digital images may be generated by redirecting or splitting the optical beam path to an imaging system that outputs digital data representing the digital image to a display for output to the user, such as through an ocular of the surgical microscope.

The above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. A method for displaying images during surgery, the method comprising:
displaying an analog image of a surgical site to a user using a surgical microscope, wherein the analog image comprises light from an objective lens of the surgical microscope;
receiving a first indication to display a digital image of the surgical site using the surgical microscope;
responsive to the first indication, redirecting the light from the objective lens to an imaging system enabled to acquire the digital image; and
displaying the digital image of the surgical site to the user using a display device.

2. The method of claim 1, further comprising:
performing digital processing on the digital image, the digital processing including at least one operation selected from:
changing the contrast of the digital image;
changing the brightness of the digital image;
annotating the digital image with text annotations;
annotating the digital image with a measurement display; and
annotating the digital image with a digital marker.

3. The method of claim 1, further comprising:
receiving a second indication to display the analog image of the surgical site using the surgical microscope; and
responsive to the second indication, redirecting the light to an ocular of the surgical microscope; and
displaying the analog image in the ocular.

4. The method of claim 1, wherein redirecting light from the objective lens further comprises:
controlling a first mirror shutter in an optical path of the light to redirect the light to the imaging system; and
controlling a second mirror shutter in the optical path to redirect the digital image to the ocular.

5. The method of claim 3, wherein redirecting light to the ocular further comprises:
controlling a first mirror shutter in an optical path of the light to redirect the light to the ocular; and
controlling a second mirror shutter in the optical path to redirect the light to the ocular.

6. The method of claim 1, wherein redirecting light from the objective lens further comprises:
using a beam splitter in an optical path of the light to split the light between the imaging system and the optical path;
using a beam combiner in the optical path to superimpose the digital image to the optical path;
controlling a shutter between the beam splitter and the beam combiner to control propagation of the light from the objective lens; and
controlling power to the display device for displaying the digital image.

7. A surgical microscope for displaying images during surgery, the surgical microscope comprising:
a controller enabled to control redirection of light from an objective lens of the surgical microscope for display to a user, the controller further enabled to:
display an analog image of a surgical site to the user, wherein the analog image comprises light from the objective lens;
receive a first indication to display a digital image of the surgical site;
responsive to the first indication, redirect the light from the objective lens to an imaging system enabled to acquire the digital image; and
display the digital image of the surgical site to the user using a display device.

8. The surgical microscope of claim 7, wherein the controller is further enabled to:
perform digital processing on the digital image, the digital processing including at least one operation selected from:
changing the contrast of the digital image;
changing the brightness of the digital image;
annotating the digital image with text annotations;
annotating the digital image with a measurement display; and
annotating the digital image with a digital marker.

9. The surgical microscope of claim 7, wherein the controller is further enabled to:
receive a second indication to display the analog image of the surgical site using the surgical microscope; and
responsive to the second indication, redirect the light to an ocular of the surgical microscope; and
display the analog image in the ocular.

10. The surgical microscope of claim 9, wherein redirecting light from the objective lens further comprises:
controlling a first mirror shutter in an optical path of the light to redirect the light to the imaging system; and
controlling a second mirror shutter in the optical path to redirect the digital image to the ocular.

11. The surgical microscope of claim 9, wherein redirecting light to the ocular further comprises:
controlling a first mirror shutter in an optical path of the light to redirect the light to the ocular; and
controlling a second mirror shutter in the optical path to redirect the light to the ocular.

12. The surgical microscope of claim 9, wherein redirecting light from the objective lens further comprises:
using a beam splitter in an optical path of the light to split the light between the imaging system and the optical path; and
using a beam combiner in the optical path to superimpose the digital image to the optical path;
controlling a shutter between the beam splitter and the beam combiner to control propagation of the light from the objective lens; and
controlling power to the display device for displaying the digital image.

13. A controller for a surgical microscope for displaying images during surgery, the controller enabled to:
display an analog image of a surgical site to the user, wherein the analog image comprises light from the objective lens;
receive a first indication to display a digital image of the surgical site;
responsive to the first indication, redirect the light from the objective lens to an imaging system enabled to acquire the digital image; and
display the digital image of the surgical site to the user using a display device.

14. The controller of claim 13, further enabled to:
perform digital processing on the digital image, the digital processing including at least one operation selected from:
changing the contrast of the digital image;
changing the brightness of the digital image;
annotating the digital image with text annotations;
annotating the digital image with a measurement display; and
annotating the digital image with a digital marker.

15. The controller of claim 13, further enabled to:
receive a second indication to display the analog image of the surgical site using the surgical microscope; and
responsive to the second indication, redirect the light to an ocular of the surgical microscope; and
display the analog image in the ocular.

16. The controller of claim 15, wherein redirecting light from the objective lens further comprises:
controlling a first mirror shutter in an optical path of the light to redirect the light to the imaging system; and
controlling a second mirror shutter in the optical path to redirect the digital image to the ocular.

17. The controller of claim 15, wherein redirecting light to the ocular further comprises:
controlling a first mirror shutter in an optical path of the light to redirect the light to the ocular; and
controlling a second mirror shutter in the optical path to redirect the light to the ocular.

18. The controller of claim 15, wherein redirecting light from the objective lens further comprises:
using a beam splitter in an optical path of the light to split the light between the imaging system and the optical path; and
using a beam combiner in the optical path to superimpose the digital image to the optical path;
controlling a shutter between the beam splitter and the beam combiner to control propagation of the light from the objective lens; and
controlling power to the display device for displaying the digital image.
